(19) United States
(12) Patent Application Publication
(10) Pub. No.: US 2008/0118473 A1
(43) Pub. Date: May 22, 2008

(54) Methods of treating a respiratory condition comprising probiotic treatment

(75) Inventors: Barry Pius Kiely, Passage West (IE); Liam Diarmuid O'Mahony, Rathcormac (IE); David Alexander Lawson, Glendale, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignees: The Procter & Gamble Company, Cincinnati, OH (US); Alimentary Health Ltd., County Cork (IE)

(21) Appl. No.: 11/981,035

(22) Filed: Oct. 31, 2007

Related U.S. Application Data
(60) Provisional application No. 60/855,861, filed on Nov. 1, 2006.

(51) Int. Cl. A61K 35/74; A61P 11/00; A61P 31/16

(52) U.S. Cl. 424/93.4

(57) ABSTRACT

Disclosed herein are methods for treating a respiratory condition in a mammal, such as a human. The methods employ use of a composition comprising a Bifidobacterium probiotic.

### CROSS REFERENCE TO RELATED APPLICATIONS

This application claims the benefit of U.S. Provisional Application No. 60/855,861, filed Nov. 1, 2006.

### FIELD OF THE INVENTION

The present invention is directed to the field of treatment of a respiratory condition, wherein a composition comprising a Bifidobacterium probiotic is employed.

### BACKGROUND OF THE INVENTION

Respiratory conditions can be triggered by any of a variety of sources including allergens or pathogens of viral, fungal or bacterial origin.

As an example, without limitation by theory, allergy may be the result of hyper-reactivity of the immune system to foreign or self antigens. Type I allergy, such as allergic rhinitis (e.g., hay fever) or atopic dermatitis, occurs in allergic subjects upon exposure to environmental allergens (e.g., pollens or dust mites), and results in key clinical symptoms such as sore throat, cough, fatigue, sneeze, running nose, stuffy nose, itchy nose, itchy eyes, and watery eyes. In healthy status, the immune system maintains a balance between cytokines produced by different helper T lymphocyte subsets: Th1 and Th2 lymphocytes. In contrast, an allergic subject demonstrates a biased dysfunction of Th2 over Th1 that leads to an elevated IgE antibody production. IgE antibody-bound mast cells interact with allergen, triggering release of chemical mediators (e.g., histamine, leukotriene) and cause vasodilation and hypersecretion in various tissues. Antihistamines or leukotriene antagonists compete with the secreted inflammatory mediators from mast cells and significantly reduce clinical respiratory symptoms.

The elevated production of IgE may be induced by hyper-reactivity of Th2 lymphocytes that secrete cytokines (e.g., IL-4, IL-5). It has been shown that down-regulation of Th2 cytokines can improve reduction in clinical respiratory symptoms. In addition Th1 cytokines (e.g., interferon-gamma, IL-12) may counteract Th2 cytokines and regain healthy state in murine systems. Rengaranjan et al., “Transcriptional Regulation of Th1/Th2 Polarization”, Immunology Today, Vol. 21, pp. 479-483 (2000) and Swain, S. L., “Helper T Cell Differentiation”, Current Opinion in Immunology, Vol. 11, pp. 180-185 (1999).

Certain Lactobacillus probiotics have been shown to modulate immune response by stimulation of Th1 cytokine production and hence modulate excessive Th2 reactivity. Niers et al., “Identification of Strong Interleukin-10 Producing Lactic Acid Bacteria which Down-regulate T Helper Type-2 Cytokines”, Clin. Exp. Allergy, Vol. 35, pp. 1481-1489 (2001). However, it has been further demonstrated that not all Lactobacillus are similar, or have, efficacy to confer immune normalization. Fujiwara et al., “The Anti-allergic Effect of Lactic Acid Bacteria are Strain Dependent and Mediated by Effects on Both Th1/Th2 Cytokine Expression and Balance”, Int. Arch. Allergy Immunol., Vol. 135, pp. 205-215 (2004).

Weak efficacy has been shown by probiotics for the relief of symptoms in clinical trials for allergic rhinitis, in certain instances limited only to quality of life rather than alleviation of respiratory symptoms or the condition itself. Ishida et al., “Clinical Effect of Lactobacillus acidophilus Strain L-92 on Perennial Allergic Rhinitis: A Double-blind, Placebo-controlled Study”, J. Dairy Sci., Vol. 88, pp. 527-533 (2005); Wang et al., “Treatment of Perennial Allergic Rhinitis with Lactic Acid Bacteria”, Pediatr. Allergy Immunol., Vol. 15, pp. 152-158 (2004); and Xiao et al., “Effect of Probiotic Bifidobacterium longum Bb-536 in Relieving Clinical Symptoms and Modulating Plasma Cytokine Levels in Japanese Cedar Pollinosis During the Pollen Season. A Randomized Double-blind, Placebo-controlled Trial”, J. Investig. Allergo Clin. Immunol., Vol. 16, pp. 86-93 (2006).

There is therefore an unmet need for methods of treating respiratory conditions using a composition containing a probiotic. The present invention meets this need.

### SUMMARY OF THE INVENTION

The present invention is directed to methods of treating a respiratory condition in a mammal, comprising orally administering to the mammal a composition comprising a strain of Bifidobacterium isolated from resected and washed mammalian gastrointestinal tract.

### DETAILED DESCRIPTION OF THE INVENTION

Various documents including, for example, publications and patents, are recited throughout this disclosure. All documents are hereby incorporated by reference.

All percentages and ratios are calculated by weight unless otherwise indicated. All percentages and ratios are calculated based on the total composition unless otherwise indicated.

Referenced herein may be trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments and/or individual components are disclosed. As will be apparent to the ordinarily skilled practitioner, all combinations of such embodiments and components are possible and can result in preferred executions of the present invention.

The compositions herein may comprise, consist essentially of, or consist of any of the components as described herein.

While various embodiments and individual components of the present invention have been illustrated and described, various other changes and modifications can be made without departing from the spirit and scope of the invention. As will be also be apparent, all combinations of the embodiments and components taught in the foregoing disclosure are possible and can result in preferred executions of the invention.

### METHOD OF THE PRESENT INVENTION

The methods of the present invention comprise orally administering (i.e., through ingestion) a composition of the present invention to a mammal to treat a respiratory condition. The composition contains a probiotic as described herein. In one embodiment, the mammal is a human, or may be a domestic animal such as a cats, dogs, cows, rabbits, or horses. In one embodiment, the mammal is a human. In another embodiment, the mammal is a cat or a dog.

The respiratory condition treated through this invention will be well understood by one of ordinary skill in the art. For example, the respiratory condition may be a respiratory viral condition such as common cold, influenza, coronavirus, parainfluenza virus, respiratory syncytial virus, viral pneumonia, or infectious pharyngotracheitis virus-induced illnesses. As another example, the respiratory condition may be a respiratory bacterial condition such as Hemophilus influenzae, mycobacteria, pasteurella, Pneumocystis jiroveci, Mycobacterium tuberculosis, Streptococcus pneumoniae, bacterial pneumonia, or Klebsiella pneumoniae. As another example, the respiratory condition may be a respiratory fungal condition such as aspergillosis, histoplasmosis, Blastomyces, dermatitidis, Cryptococcus neoformas, Coccidioidomycosis, or Pneumocystis jiroveci. As another example, the respiratory condition may be allergy, such as allergy to pollen, fungi, or environmental allergens. As another example, the respiratory condition may be asthma, rhinitis, sinusitis, bronchiolitis, acute respiratory distress syndrome (ARDS), severe acute respiratory syndrome (SARS), respiratory cancer, or conditions pursuant to respiratory surgeries (pre- and post-operative management). In one embodiment, the respiratory condition is selected from common cold, influenza, allergy, rhinitis, or sinusitis. As used herein, treatment of a respiratory condition means the prevention, cure, mitigation, or alleviation of that condition, or the prevention, cure, mitigation or alleviation of any, some, or all of the symptoms associated with that condition. Symptoms may include, for example, sore throat, cough, fatigue, sneeze, running nose, stuffy nose, itchy nose, itchy eyes, watery eyes, and combinations thereof.

Treatment of the respiratory condition may additionally or alternatively mean treatment of symptoms or regulation of body systems associated with any of the foregoing respiratory conditions. For example, treatment of the respiratory condition may include such benefits as: improved quality of life or better health through allergy season, cold season, influenza season, and/or the like; improved respiratory health through allergy season, cold season, influenza season, and/or the like; prevention, cure, mitigation or alleviation of symptoms through allergy season, cold season, influenza season, and/or the like; normal ability to perform daily tasks through allergy season, cold season, influenza season, and/or the like; strengthening natural defenses and/or immunity during allergy season, cold season, influenza season, and/or the like; or support natural defenses and/or maintain immune system during allergy season, cold season, influenza season, and/or the like.

While known to those ordinarily skilled in the art, for some convenience various methods of analysis directed to these benefits are described herein below.

As used herein, the term “orally administering” with respect to the mammal means that the mammal ingests or a human is directed to administer, or does administer, oneself (or another human or other animal) one or more compositions herein. Wherein the human is directed to administer the composition, such direction may be that which instructs and/or informs the human that use of the composition may and/or will provide the referenced benefit, for example, alleviation of one or more symptoms associated with the common cold or influenza. For example, such direction may be oral direction (e.g., through oral instruction from, for example, a physician, pharmacist, nurse, veterinarian or other health professional), radio or television media (i.e., advertisement), or written direction (e.g., through written direction from, for example, a veterinarian or other health professional (e.g., scripts), sales professional or organization (e.g., through, for example, marketing brochures, pamphlets, or other instructive paraphernalia), written media (e.g., internet, electronic mail, or other computer-related media)), and/or packaging associated with the composition (e.g., a label present on a container holding the composition). As used herein, “written” means through words, pictures, symbols, and/or other visible descriptors. Such information need not utilize the actual words used herein, for example, “respiratory”, “mammal”, “human”, or “treatment”, but rather use of words, pictures, symbols, and the like conveying the same or similar meaning are contemplated within the scope of this invention.

The compositions described herein may be orally administered in any convenient form including, for example, a capsule, tablet (including swallowable or chewable forms), suspension, powders (including such powders which are suitable for admixture with a liquid such as, for example, water or milk), or the like. In one embodiment herein, the composition is selected from tablets, capsules, and mixtures thereof. The compositions described herein may be used as a supplement to ordinary diet (e.g., a dietary supplement) or may also serve as food for the mammal (e.g., a yogurt or other dairy product).

Administration may be on an as-needed or as-desired basis, for example, once-monthly, once-weekly, or daily (including multiple times daily). When utilized as a supplement to ordinary diet, the composition may be administered directly to the mammal (e.g., a capsule or tablet) or otherwise contacted with or admixed with food (e.g., mixed with yogurt or milk). The amount of composition utilized may be dependent on a variety of factors, including the health status of the mammal, age, gender, or other like factors of ordinary consideration.

The methods of the present invention utilize administration of a probiotic strain of Bifidobacterium. Probiotics are microorganisms, either viable or dead, processed compositions of micro-organisms, their constituents such as proteins or carbohydrates, or purified fractions of bacterial ferments that beneficially affect a host. The general use of probiotics is in the form of viable cells. However, it can be extended to non-viable cells such as killed cultures or compositions containing beneficial factors expressed by the probiotic. This may include thermally killed microorganisms, or microorganisms killed by exposure to altered pH or subjected to pressure. For the purpose of the present invention, “probiotic” is further intended to include the metabolites generated by the microorganisms during fermentation, if they are not separately indicated. These metabolites may be released to the medium of fermentation, or they may be stored within the microorganism. As used herein “probiotic” also includes bacteria, bacterial homogenates, bacterial proteins, bacterial extracts, bacterial ferment supernatants, and mixtures thereof, which perform beneficial functions to the host animal when given at a therapeutic dose.

The composition utilized in the method herein comprise a strain of Bifidobacterium probiotic isolated from resected and washed mammalian gastrointestinal tract, such as those as disclosed in WO 00/42168. The mammalian gastrointestinal tract may be, for example, human or domestic animal (e.g., cat, dog, cow, rabbit, or horse) gastrointestinal tract. The mammal treated and the mammalian source of Bifidobacterium isolation may be, but need not be, independent. For example, one embodiment of the invention includes treatment of a respiratory condition in a human comprising orally administering a strain of Bifidobacterium isolated from resected and washed human gastrointestinal tract.

It is found that lactic acid bacteria of the genus Bifidobacteria obtainable by isolation directly from resected and washed gastrointestinal tract of mammals are adherent to the gastrointestinal tract following feeding of viable bacterial cells, and are also significantly immunomodulatory when administered to a mammal in viable, non-viable or fractionated form. Without being bound by theory, it is believed that Bifidobacteria obtainable by isolation from resected and washed gastrointestinal tract closely associate with the gut mucosal tissues. This is believed to result in the probiotic Bifidobacteria used herein generating alternative host responses that result in its probiotic action. It has been found that the Bifidobacteria obtainable by isolation from resected and washed gastrointestinal tract can modulate the host's immune system via direct interaction with the mucosal epithelium, and the host's immune cells. This immunomodulation, in conjunction with the traditional mechanism of action associated with a probiotic, i.e., the prevention of pathogen adherence to the gut by occlusion and competition for nutrients, results in the Bifidobacteria of the present invention being highly efficacious as a probiotic organism.

In one embodiment, the Bifidobacterium herein is able to maintain viability following transit through the gastrointestinal tract. This is desirable in order for live cultures of the bacteria to be taken orally, and for colonization to occur in the intestines and bowel following transit through the esophagus and stomach. Colonization of the intestine and bowel by the probiotic is desirable for long term probiotic benefits to be delivered to the host. Oral administration of non-viable cells or purified isolates thereof may induce temporary benefits, but as the bacteria are not viable, they are not able to grow, and are more limited in ability to continuously deliver a probiotic effect. As a result this may require the host to be dosed regularly in order to maintain the health benefits. In contrast, viable cells that are able to survive gastric transit in the viable form, and subsequently colonize by adhering to and proliferating on the gut mucosa are better able to deliver probiotic effects continuously.

In one embodiment, the strain of Bifidobacterium is of a species selected from the group consisting of Bifidobacterium longum, Bifidobacterium animalis, Bifidobacterium adolescentis, Bifidobacterium bifidum, Bifidobacterium infantis, Bifidobacterium thermophilum, Bifidobacterium lactis, and mixtures thereof.

In one embodiment herein, the probiotic is a Bifidobacterium infantis strain. An example includes Bifidobacterium infantis strain designated UCC35624, described in WO 00/42168 as being deposited at the National Collections of Industrial and Marine Bacteria Ltd (NCIMB) on Jan. 13, 1999, and accorded the accession number NCIMB 41003.

In one embodiment, the methods herein comprise administration of a therapeutically effective amount of the Bifidobacterium. As used herein, the term “therapeutically effective amount” with reference to the Bifidobacterium, means that amount of the bacteria sufficient to provide the desired effect or benefit to a host animal in need of treatment, yet low enough to avoid adverse effects such as toxicity, irritation, or allergic response, commensurate with a reasonable benefit/risk ratio when used in the manner of the present invention. The specific “therapeutically effective amount” will vary with such factors as the particular condition being treated, the physical condition of the user, the duration of the treatment, the nature of concurrent therapy (if any), the specific dosage form to be used, the carrier employed, the solubility of the dose form, and the particular dosing regimen.

In one embodiment herein, the compositions used in the methods herein comprise from about 10⁴ to about 10¹⁴ CFU of the Bifidobacterium, in another embodiment from about 10⁶ to about 10¹² CFU of the Bifidobacterium, in another embodiment from about from about 10⁸ to about 10¹⁰ CFU of the Bifidobacterium. As used herein, the abbreviation CFU (referring to “colony-forming unit”) designates the number of bacterial cells revealed by microbiological counts on agar plates, as will be commonly understood in the art. The Bifidobacterium may be administered in either viable form, or as killed cells, or distillates, isolates or other fractions of the fermentation products of the Bifidobacterium used herein, or any mixture thereof.

Non-limiting examples of compositions suitable for the treatment of respiratory tract conditions include any product commercially available under the VICKS®, NYQUIL®, or DAYQUIL® trade names (all commercially available from The Procter & Gamble Company), or any product containing pseudoephedrine, phenylephrine, chlorpheniramine, dextromethorphan, diphenhydramine, doxylamine, guaifenesin, or the like (see, for example, U.S. Pat. No. 5,025,019), with the addition that such product contains a strain of Bifidobacterium isolated from resected and washed mammalian gastrointestinal tract.

The compositions herein may, independently, comprise one or more additional optional components to enhance their performance. For example, one or more prebiotics, plasticizers, coloring agents, flavoring agents, sweeteners, anti-oxidants, buffering agents, slip aids, other excipients, and the like can be optionally included in the compositions herein. Non-limiting examples of optional components are given below:

### Fiber

The compositions comprising the Bifidobacterium used herein may also comprise a fiber, such as prebiotic. “Prebiotic” includes substances or compounds that are fermented by the intestinal flora of a mammal and hence promote the growth or development of probiotic bacteria in the gastrointestinal tract of the mammal at the expense of pathogenic bacteria.

Illustrative soluble fibers which may optionally be used singularly or in combination in all embodiments of the present invention include but are not limited to pectins, psyllium, guar gum, xanthan gum, alginates, gum arabic, fructo-oligosaccharides, inulin, agar, and carrageenan.

In one embodiment, the soluble fibers for use herein are glucose polymers, preferably those which have branched chains. Preferred among these soluble fibers is one marketed under the trade name Fi□ersol2, commercially available from Matsutani Chemical Industry Co., Itami City, Hyogo, Japan.

Pectins are also useful soluble fibers herein. Pectins may be obtained by hot acidic extraction from citrus peels and may be obtained, for example, from Danisco Co., Braband, Denmark.

Other non-limiting examples of suitable fibers include oligosaccharides, such as inulin and its hydrolysis products commonly known as fructooligosaccharides, galacto-oligosaccarides, xylo-oligosaccharides or oligo derivatives of starch. The fibers may be provided in any suitable form. For example, the fibers may be provided in the form of plant material which contains the fiber. Suitable plant materials include asparagus, artichokes, onions, wheat or chicory, or residues of these plant materials. Alternatively, the fiber may be provided as an inulin extract, for example extracts from chicory are suitable. Suitable inulin extracts may be obtained from Orafti SA of Tirlemont 3300, Belgium under the trademark “RAFTILINE”. Alternatively, the fiber may be in the form of a fructooligosaccharide such as obtained from Orafti SA of Tirlemont 3300, Belgium under the trademark “RAFTILOSE”. Otherwise, the fructooligosaccharides may be obtained by hydrolyzing inulin, by enzymatic methods, or by using microorganisms.

### Plasticizers

Without intending to be limited by theory, plasticizers cause a composition to become more easily deformed, less brittle, or less prone to mechanical damage. Thus, one or more plasticizers may optionally be added to the present compositions, particularly a portion of the composition such that the composition is not susceptible to cracking (creation of voids).

Non-limiting examples of plasticizers include phthalates (e.g., diethyl phthalate, dibutyl phthalate, dioctyl phthalate), citrates (e.g., triethyl citrate (e.g., CITROFLEX 2), acetyl triethyl citrate, tributyl citrate, and acetyl tributyl citrate), polyhydric alcohols, (e.g., sorbitol, glycerol), triacetin (glyceryl triacetate), polyethylene glycol (e.g., CARBOWAX 400), polysorbate 80, acetylated monoglycerides, glycerol, propylene glycol, fatty acid esters, surfactant polymers, camphor, silicone oil, castor oil, and mixtures thereof.

The amount of plasticizer used will vary, for example depending on the plasticizer used and the desired character of the composition. For example, in one embodiment a composition comprising a plasticizer may comprise from about 0.001% to about 20%, or from about 0.01% to about 15% plasticizer, and or from about 0.1% to about 10% of the plasticizer, all by weight of the composition.

### Coloring Agents

One or more pigments or other suitable coloring agents, such as dyes and lakes, may be incorporated into the compositions. For example, U.S. FD&C dyes (e.g., yellow #5, blue #2, red # 40) or U.S. FD&C lakes may be used. Illustrative lakes which may be used in the present invention include, for example, Lake red #40, yellow #6, blue #1, and the like. Additionally, a mixture of U.S. FD&C dyes and/or U.S. FD&C lakes in combination with other conventional food and food colorants may be used. As further examples, Riboflavin and □-carotene may also be used. Additionally, other natural coloring agents may be utilized including, for example, fruit, vegetable, or plant extracts such as grape, black currant, aronia, carrot, beetroot, red cabbage, and hibiscus.

The amount of coloring agent used will vary, depending on the agents used and the character or intensity desired in the finished composition. One of ordinary skill in the art will readily make such determination.

### Flavoring Agents

One or more flavoring agents may be incorporated in the compositions herein in order to enhance their palatability. Any natural or synthetic flavor agent can be used in the present invention. As used herein, such flavors may be synthetic or natural flavors.

For example, one or more botanical or fruit flavors may be utilized herein. Illustrative fruit flavors are exotic and lactonic flavors such as, for example, passion fruit flavors, mango flavors, pineapple flavors, cupuacu flavors, guava flavors, cocoa flavors, papaya flavors, peach flavors, and apricot flavors. Besides these flavors, a variety of other fruit flavors can be utilized such as, for example, apple flavors, citrus flavors, grape flavors, raspberry flavors, cranberry flavors, cherry flavors, grapefruit flavors, and the like. These fruit flavors can be derived from natural sources such as fruit juices and flavor oils, or may alternatively be synthetically prepared.

The amount of flavoring agent used will vary, depending on the agents used and the character or intensity desired in the finished composition. One of ordinary skill in the art will readily make such determination.

### Sweeteners

One or more sweeteners, including for example carbohydrate sweeteners and natural or artificial no/low calorie sweeteners may optionally be used herein. For example, the compositions used herein can be sweetened with any of the carbohydrate sweeteners, such as monosaccharides or disaccharides. Illustrative sugar sweeteners for use in the compositions of the present invention are sucrose, fructose, glucose, maltose, and mixtures thereof.

One or more high intensity sweeteners may be utilized. For example, one or more of the following sweeteners may be utilized: saccharin, cyclamates, L-aspartyl-L-phenylalanine lower alkyl ester sweeteners (e.g., aspartame); L-aspartyl-D-alanine amides disclosed in U.S. Pat. No. 4,411,925; L-aspartyl-D-serine amides disclosed in U.S. Pat. No. 4,399,163; L-aspartyl-L-1-hydroxymethylalkaneamide sweeteners disclosed in U.S. Pat. No. 4,338,346; L-aspartyl-1-hydroxyethyalkaneamide sweeteners disclosed in U.S. Pat. No. 4,423,029; L-aspartyl-D-phenylglycine ester and amide sweeteners disclosed in European Patent Application 168,112, published Jan. 15, 1986; N—[N-3,3-dimethylbutyl)-L-alpha-aspartyl]-L-phenylalanine 1-methyl ester sweeteners disclosed in WO 99/30576; thaumatin; dihydrochalcones; cyclamates; steviosides; glycyrrhizins, synthetic alkoxy aromatics; sucralose; suosan; miraculin; monellin; sorbitol, xylitol; talin; cyclohexylsulfamates; substituted imidazolines; synthetic sulfamic acids such as acesulfame, acesulfame K and n-substituted sulfamic acids; oximes such as perilartine; peptides such as aspartyl malonates and succanilic acids; dipeptides; amino acid based sweeteners such as gem-diaminoalkanes, meta-aminobenzoic acid, L-aminodicarboxylic acid alkanes, and amides of certain alpha-aminodicarboxylic acids and gem-diamines; and 3-hydroxy-4-alkyloxyphenyl aliphatic carboxylates or heterocyclic aromatic carboxylates; erythritol; and mixtures thereof. Aspartame is particularly preferred.

The amount of sweetener used will vary, depending on the agents used and the character or intensity desired in the finished composition. One of ordinary skill in the art will readily make such determination.

### Anti-Oxidants

One or more anti-oxidants may be utilized in the compositions of the present invention. Naturally occurring as well as synthetic anti-oxidants may be used. Non-limiting examples of natural anti-oxidants include tocopherols (e.g., vitamin E), ascorbic acid (e.g., vitamin C), vitamin A (e.g., beta-carotene), grape seed extract, selenium, and coenzyme Q10. Non-limiting examples of synthetic anti-oxidants include butylated hydroxytoluene (BHT), butylated hydroxyanisole (BHA), and propyl gallate.

### Buffering Agents

One or more buffering agents may be utilized in the compositions of the present invention in order to, for example, maintain a constant pH within an environment. For example, acetate buffers, citrate buffers, and phosphate buffers may be used. Non-limiting examples include acetic acid, sodium acetate, citric acid, sodium citrate, monobasic sodium phosphate, dibasic sodium phosphate, and sodium chloride.

### Slip Aids

One or more slip aids may optionally be included in the present compositions to improve surface friction, water resistance, abrasion resistance, or other mechanical properties of the composition. For example, a slip aid may be included on the surface of the composition, such that a mammal can more easily swallow the composition when orally administered.

Non-limiting examples of slip aids that may be used include wax additives including, for example, animal, fossil, vegetable, mineral, or synthetic waxes. Preferred wax additives include carnuba, beeswax, carob, candelilla, ozocerite, polyethylene waxes, paraffin waxes, polypropylene waxes, and the like. Other non-limiting examples include surfactants, glycerin, oils, and polyethylene glycols.

The amount of slip aid used will vary, depending on the aid used and the specific purpose of the aid. One of ordinary skill in the art will readily make such determination.

### Printed Material

The compositions herein may optionally comprise printed material. For example, the composition may comprise text, words, pictures, symbols, or other visible images that may or may not convey useful information to the manufacturer or consumer. To illustrate, a capsule may indicate the identity or dosage level of the probiotic, or may indicate a trademark or other like descriptor. Material may be printed on the surface of a composition herein via a variety of well-known methods, for example, passing a capsule over an embossed roller which transfers the material to the capsule.

### Methods of Analysis

The present compositions may be utilized to treat a respiratory condition in a mammal. Various methods of demonstrating such treatments are well-known to those of ordinary skill in the art. As examples, the following provides illustrations of certain methods which may be used. These methods are not intended to limit the scope of the invention.

Treatment of the respiratory condition may be measured using techniques known to those skilled in the art.

Such techniques include in vivo techniques such as monitoring cytokines, chemokines, inflammatory cells (neutrophil, monocyte, or eosinophil) in the respiratory tract of a mammalian subject.

Such techniques may further include in vitro techniques such as monitoring cytokines (interferon-gamma, tumor necrosis factor-alpha, IL1, IL2, and IL6) and chemokines (IP10, RANTES, ITAC, IL8, MMPs, MIPs, MCPs) from respiratory lavage cells or peripheral blood cells with and without stimulation.

Such techniques may further include in vivo techniques to measure an allergen sensitized mammals with a respiratory challenge with allergen or a pharmacological agent (e.g., histamine or methacholine).

For example, systemic activity relevant to the respiratory condition may be detected and monitored using in vivo immune function tests such as lymphocyte blastogenesis, natural killer cell activity, antibody response to vaccines, delayed-type hypersensitivity, and mixtures thereof. Such methods are briefly described herein, but well known to those skilled in the art.
1. Lymphocyte blastogenesis: This assay measures the proliferative response in vitro of lymphocytes isolated from fresh whole blood of test and control animals to various mitogens and is a measure of overall T- and B-cell function. Briefly, peripheral blood mononucleocytes (PBMC) are isolated from whole blood by Ficoll-Hypaque density centrifugation methods known to those skilled in the art. The isolated PBMCs are washed twice in RPMI 1640 cell media supplemented with HEPES, L-glutamine and penicillin/streptomycin. The washed cells are resuspended in RPMI 1640, counted, and the cell density adjusted appropriately. The 2×10⁵ cells are exposed to a range of concentrations (0.1 □g/ml to 100 □g/ml) of various mitogens, some examples of which include pokeweed mitogen (Gibco), phytohaemagglutinin (Gibco) and conconavalin A (Sigma) in triplicate for 72 hours at 37° C. and 5% CO₂ with 10% foetal bovine serum (Sigma). At 54 hours the cells are pulsed with 1□Ci ³H-thymidine, and the cells harvested and scintillation counts read on a TopCount NXT at 72 hours.
2. Natural killer cell activity: As described in U.S. Pat. No. 6,310,090, this assay measures the in vitro effector activity of natural killer cells isolated from fresh whole blood of test and control animals. Natural killer cells are a component of the innate immune function of a mammal. Feline thyroid adenocarcinoma cells are used as target cells in assessing NK cell cytotoxic activity. This cell line is previously shown to be susceptible to killing by feline NK cell. Target cells are cultured in a T75 flask with 20 mL minimum essential medium (MEM; Sigma Chem. Co., St. Louis, Mo.) supplemented with 10% fetal calf serum (FCS), 100 U/mL of penicillin and 100 □g/mL of streptomycin. When confluent, target cells are trypsinized, washed 3 times and resuspended to 5×10⁵ cells/mL in complete medium (RPMI-1640+10% FCS+100 U/mL of penicillin+100 □g/mL of streptomycin). Triplicate 100 microliter aliquots of the target cells are pipetted into 96-well U-bottom plates (Costar, Cambridge, Mass.) and incubated for 8 hours to allow cell adherence. Lymphocytes (effector cells; 100 microliters) isolated by Ficoll-Hypaque separation (as described above) are then added to the target cells to provide an effector/target cell (E:T) ratio of 10:1. After 10 hours of incubation at 37° C., 20 microliters of a substrate containing 5 micrograms of 3-(4,5-dimethylthiazol-2-yl)-2,5-diphenyltetrazolium bromide (MTT) is added. The mixture is incubated for 4 hours at 37° C. after which the unmetabolized MTT is removed by aspiration. The formazan crystals are dissolved by adding 200 microliters of 95% ethanol. Optical density is measured at 570 nm using a microplate reader. The percentage of NK cell-specific lysis is calculated as follows:

Specific Cytotoxicity (%)=100×{1-[(OD of target cells and effector cells−OD of effector cells)/(OD of target cells)]}
Probiotic treatment may potentiate the NK activity and enhance the host first-line defense.
3. Antibody response to vaccines: The test subjects are given an array (up to 5) of vaccines after at least 12 weeks of probiotic or control feeding. The vaccines may be a mixture of novel and redundant vaccines. The specific antibodies to the vaccines given are measured in blood for 3 weeks and the length and strength of response in control and probiotic feeding groups compared. Probiotic is anticipated to enhance antibody response for elevation of host protection.
4. Delayed-type hypersensitivity: An in vivo, non-invasive method of assessing immune system status. This test comprises an intradermal injection of the polyclonal mitogen Phytohemmaglutinin (PHA) in combination with sheep red blood cells a multivalent vaccine, histamine (100 microliters of 0.0275 g/L Histamine Phosphate; Greer, Lenoir, N.C.), or PBS (100 μL of Phosphate Buffered Saline, 8.5 g/L; Sigma). The immune response to the antigen is recorded as skinfold thickness using calipers at time intervals of 0, 24, 48 and 72 hours post-injection. An increase in skinfold thickness is indicative of a greater hypersensitivity response that should be decreased by treatment with the bacteria of the present invention.

### NON-LIMITING EXAMPLES OF THE PRESENT INVENTION

The following are non-limiting examples of the presently described methods, and the compositions employed in these methods. The described compositions are prepared utilizing conventional processes or, in the case of separate, distinct compositions may be otherwise commercially available. The examples are provided to illustrate the invention and are not intended to limit the scope thereof in any manner.

### EXAMPLE 1

A composition is prepared, having the following components in the approximate indicated amounts:

Bifidobacterium infantis UCC35624, 5% by weight of composition

Microcrystalline cellulose, 94% by weight of composition

Magnesium stearate, 1% by weight of composition

These components are filled in a hydroxypropyl methyl cellulose (HPMC) capsule.

### EXAMPLE 2

A use study to determine treatment of respiratory conditions in humans is designed, wherein the composition of Example 1 is evaluated.

Fifty (50) humans participate in the use study. The study is placed in the southwestern United States, pursuant to incidence of Cedar Fever allergy. Each human is administered one capsule of the composition of Example 1 per day. Quality of life, incidence, and symptom reduction is tracked at weeks 4, 6, 8, 10, and 12 of the study, as follows:

Self-reported perception of improved quality of life.

Self-reported perception of improved respiratory health.

Self-reported use of fewer commercially available allergy treatments.

Self-reported incidence of milder respiratory symptoms through allergy season.

Self-reported ability to regain freedom in daily tasks through allergy season.

Self-reported maintenance of normal immune system during allergy season.

Self-reported success of composition to help strengthen natural immune defenses.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating a respiratory condition in a mammal, comprising orally administering to the mammal a composition comprising a strain of Bifidobacterium isolated from resected and washed mammalian gastrointestinal tract.

2. The method according to claim 1 wherein the strain of Bifidobacterium is selected from the group consisting of Bifidobacterium longum, Bifidobacterium animalis, Bifidobacterium adolescentis, Bifidobacterium bifidum, Bifidobacterium infantis, Bifidobacterium thermophilum, Bifidobacterium lactis, and mixtures thereof.

3. The method according to claim 2 wherein the strain of Bifidobacterium is Bifidobacterium infantis.

4. The method according to claim 3 wherein the mammal is a human and the mammalian gastrointestinal tract is, independently, human gastrointestinal tract.

5. The method according to claim 4 wherein the strain of Bifidobacterium is UCC35624.

6. The method according to claim 5 wherein the respiratory condition is allergy.

7. The method according to claim 5 wherein the respiratory condition is a respiratory viral condition.

8. The method according to claim 7 wherein the respiratory condition is selected from the group consisting of common cold and influenza.

9. The method according to claim 5 which is a method of treating a symptom of the respiratory condition.

10. The method according to claim 9 wherein the symptom is selected from the group consisting of sore throat, cough, fatigue, sneeze, running nose, stuffy nose, itchy nose, itchy eyes, watery eyes, and combinations thereof.

11. The method according to claim 9 wherein the composition is selected from the group consisting of tablets, capsules, and mixtures thereof.

12. The method according to claim 11 wherein the composition comprises from about 10⁴ to about 10¹⁴ CFU of the Bifidobacterium.

13. The method according to claim 12 wherein the composition comprises from about 10⁶ to about 10¹² CFU of the Bifidobacterium.

14. The method according to claim 13 wherein the composition is orally administered daily.
